(19) United States
(12) Patent Application Publication
(10) Pub. No.: US 2009/0048491 A1
(43) Pub. Date: Feb. 19, 2009

(54) METHOD AND APPARATUS FOR LAPAROSCOPIC RETRACTION

(76) Inventors: Peter Seth Edelstein, Menlo Park, CA (US); Joseph Charles EDER, Los Altos Hills, CA (US)

Correspondence Address: GLENN PATENT GROUP, 3475 EDISON WAY, SUITE L, MENLO PARK, CA, 94025, US

(21) Appl. No.: 12/256,283

(22) Filed: Oct. 22, 2008

Related U.S. Application Data
(62) Division of application No. 11/759,746, filed on Jun. 7, 2007.
(60) Provisional application No. 60/804,408, filed on Jun. 9, 2006.

(51) Int. Cl. A61B 1/32

(52) U.S. Cl. 600/210

(57) ABSTRACT

Extraluminal and intraluminal devices and methods, and laparoscopic instrumentation, dramatically improve laparoscopic retraction of both the small and large intestine. A scaffolding external to or within a selected segment or segments of small and/or large bowel is created, allowing for the retraction of the entire selected segment or segments of bowel via grasping either the device or supported bowel wall with a laparoscopic instrument or instruments. The devices may be designed and placed in a modular fashion, with the surgeon building the final scaffolding intra-operatively, or the scaffolding may be manufactured or pre-assembled prior to intra-operative use.

### CROSS REFERENCE TO RELATED APPLICATIONS

This divisional application claims priority to U.S. Utility patent application Ser. No. 11/759,746, filed Jun. 7, 2007; which claims priority to U.S. Provisional patent application Ser. No. 60/804,408, filed Jun. 9, 2006, which document is incorporated herein in its entirety by this reference thereto.

### BACKGROUND OF THE INVENTION

1. Technical Field

The invention relates generally to laparoscopy. More particularly, the invention relates to a method and apparatus for laparoscopic retraction of organs and structures within the abdomen and pelvis.

2. Discussion of the Prior Art

Laparoscopy is a minimally invasive surgical technique that has gained widespread acceptance as the operative approach of choice for the surgical treatment of a variety of abdominal and pelvic diseases and disorders. At present, the overwhelming majority of cholecystectomy procedures, i.e. removal of the gallbladder, and a growing number of gastric, i.e. Nissen fundoplication, gastric bypass, etc., hepatic, i.e. liver procedures, and gynecologic, i.e. tubal ligation and hysterectomy procedures, are performed via a laparoscopic approach. Laparoscopic procedures addressing the small intestine (duodenum, jejunum, and ileum) and large intestine (colon and rectum) are being performed at a lower frequency, in large part to the technological and surgical difficulties associated with laparoscopic maneuvering and visualization of such long and mobile organ structures. Thus, a major contributing factor to the differing acceptances of the laparoscopic approach is anatomic. The upper abdominal structures are fixed, or relatively fixed on short or no mesenteric sheets, whereas the gastrointestinal contents filling the lower abdomen and pelvis are extremely mobile, connected to long, winding, folding mesenteric sheets and themselves many feet in length. This difference in anatomic fixation and organ length has a significant impact on surgical technique, as laparoscopic surgery is performed within the confines of the abdominal cavity when insufflated with (routinely) 1.5 to 2 liters of carbon dioxide gas. The creation of traction and counter-traction required to clearly visualize, expose, and demonstrate surgical planes, blood vessels, and other vital structures is a founding principle of all surgical procedures, regardless of approach (open or laparoscopic). The creation of adequate traction and counter-traction on fixed, short structures (such as the gallbladder) is simple whether performed via an open incision or laparoscopic approach. However, it is extremely difficult for the surgeon to adequately retract long segments of highly mobile and redundant intestine and attached mesentery via the laparoscopic approach, given the limitations of currently available instrumentation and the confined operative space. This lack of instrumentation and the confined operative space have led to the performance of “hand-assisted” laparoscopic surgery, in which the intestine is retracted during laparoscopic surgery by the surgeon's hand, itself inserted through a large, sealed port. This form of surgery, a hybrid of open and laparoscopic surgery, does not provide the patient the full benefits associated with pure laparoscopic operations, but is the current alternative for many surgeons given the absence of appropriate laparoscopic instrumentation.

Current laparoscopic instruments are capable of grasping a single point along the bowel wall. For examples, FIG. 1 shows retraction by a laparoscopic instrument at one point 10, e.g. to grasp and retract the colon, in the bowel mesentery 11 simply folds layers of bowel 12, failing to provide adequate traction for performance of the procedure. This inability to create traction and counter-traction significantly limits the ability to safely, adequately, and routinely perform laparoscopy in the small and large bowel disorders.

### SUMMARY OF THE INVENTION

The invention provides both extraluminal and intraluminal devices and methods and laparoscopic instrumentation for dramatically improving laparoscopic retraction of both the small and large intestine during the performance of laparoscopic procedures. The commonality of all the proposed devices is the creation of a scaffolding external to or within a selected segment or segments of bowel, allowing for the retraction of the entire selected segment via grasping either the device (scaffolding) or supported bowel wall with a laparoscopic instrument or instruments. The devices may be designed and placed in a modular fashion, with the surgeon building the final scaffolding intra-operatively, or the scaffolding may be manufactured or pre-assembled prior to intra-operative use.

### BRIEF DESCRIPTION OF THE DRAWINGS

FIG. 1 shows bowel retraction according to the prior art;

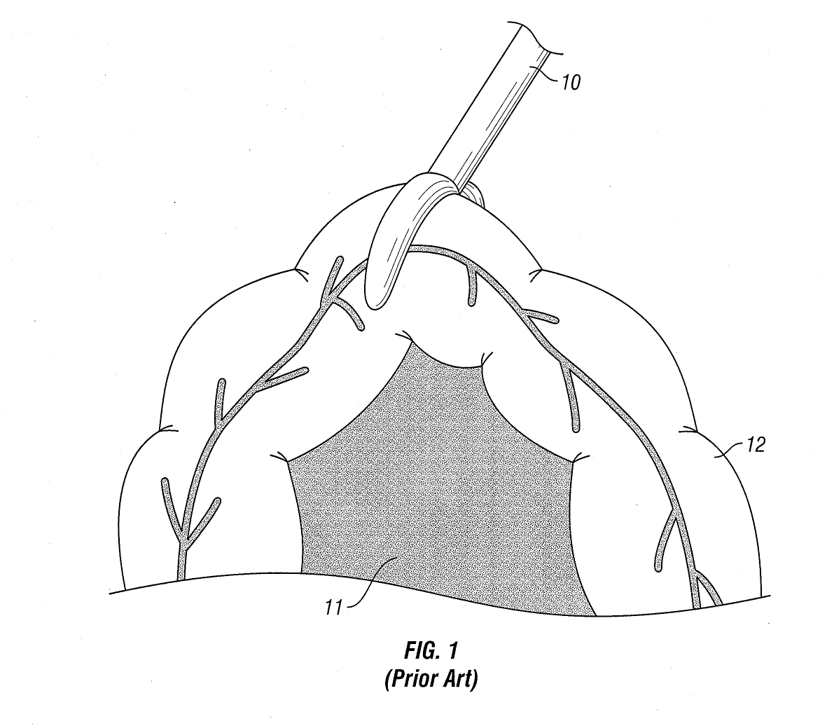

FIG. 2 shows a ring component for use in laparoscopic retraction according to the invention;

FIG. 3 shows a grasping component for use in laparoscopic retraction according to the invention;

FIG. 4 shows a cuff or sleeve for use in laparoscopic retraction according to the invention;

FIG. 5 shows a tubular or bag-like device for use in laparoscopic retraction according to the invention; and

FIG. 6 shows a laparoscopic instrument for use in laparoscopic retraction according to the invention.

### DETAILED DESCRIPTION OF A PREFERRED EMBODIMENT

The invention provides both extraluminal and intraluminal devices and methods and laparoscopic instrumentation for dramatically improving laparoscopic retraction of both the small and large intestine. The commonality of all the proposed devices is the creation of a scaffolding external to or within a selected segment or segments of bowel, allowing for the retraction of the entire selected segment via grasping either the device (scaffolding) or supported bowel wall with a laparoscopic instrument or instruments. The devices may be designed and placed in a modular fashion, with the surgeon building the final scaffolding intra-operatively, or the scaffolding may be manufactured or pre-assembled prior to intra-operative use.

### Extraluminal Devices and Methods

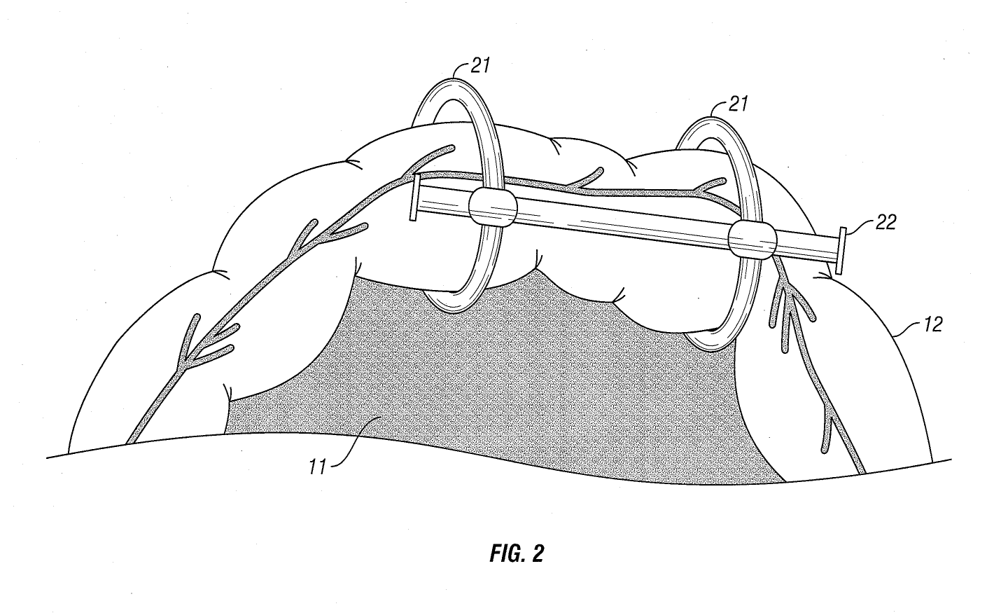

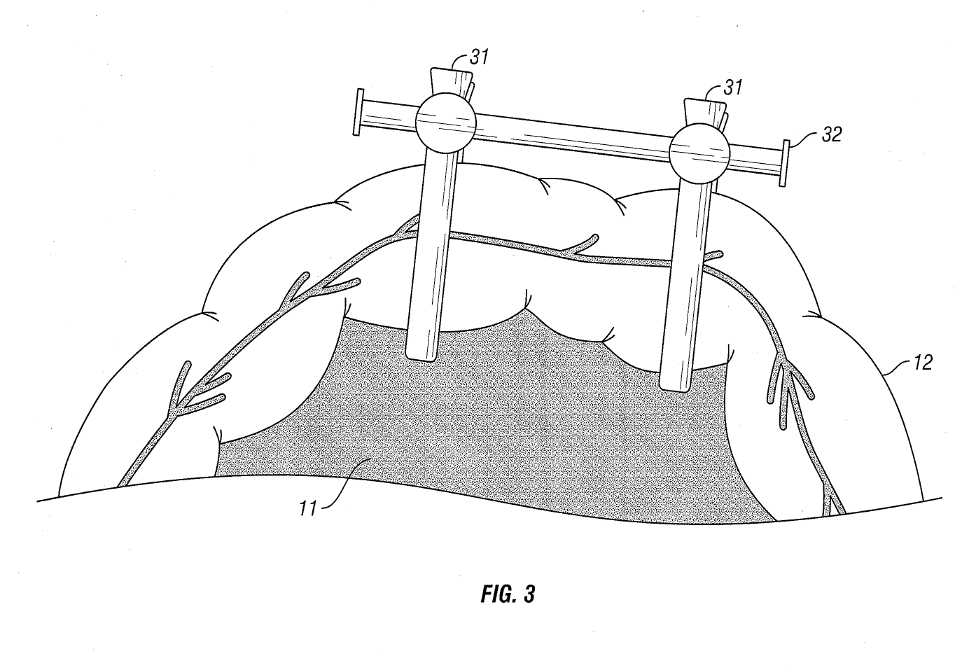

One embodiment of the invention involves the creation of a simple-to-construct extraluminal scaffolding, the components of which may be passed into the abdominal cavity via a laparoscopic or open incisional approach or transluminal or trans-orifice approach. FIGS. 2 and 3 show the use of one or more components to encircle the bowel wall, ring component 21 in FIG. 2, or grasp the bowel wall, grasping component 31 in FIG. 3, while one or more second components 22, 32, serves to interconnect and support the encircling or grasping components. The use of additional components allows the surgeon to lengthen the segment of bowel support as required by the surgical needs.

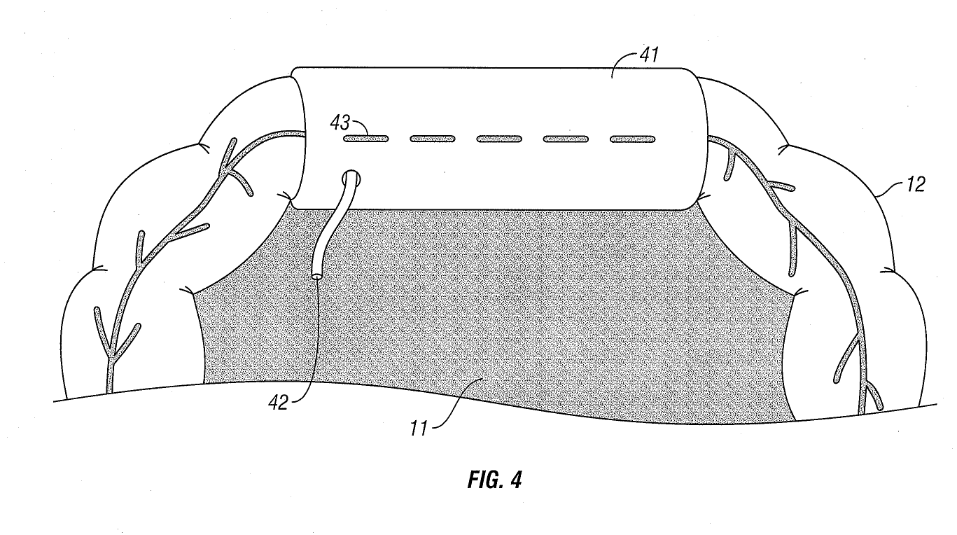

FIG. 4 shows related but different embodiment involves a cuff or sleeve 41 which may be passed into the abdominal cavity via a laparoscopic or open incisional approach or transluminal or trans-orifice approach. The cuff or sleeve may be placed along a selected section of bowel wall. Infusion of a liquid, gel, or gas, or aspiration of a liquid or gas from the device via a port 42, creates a rigid or semi-rigid scaffolding over the captured segment of bowel. The device may have flanges 43 or other means by which the support device may be grasped by a laparoscopic instrument. The use of additional lengths of cuff or sleeve allows the surgeon to lengthen the area of bowel support as required by the surgical needs.

Other extraluminal scaffolding devices can be envisioned as well.

Common to all extraluminal devices and methods is the creation of a support structure surrounding the bowel which may be manipulated via laparoscopic instrumentation and which may be lengthened as required. The scaffolding, regardless of where grasped by a laparoscopic instrument or instruments, allows for a segment or segments of bowel to be retracted using standard laparoscopic instrumentation. Retraction of a segment or segments of bowel, mimicking retraction used in open surgical procedures, allows for the performance of small and/or large bowel procedures via a laparoscopic approach.

### Intraluminal Devices and Methods

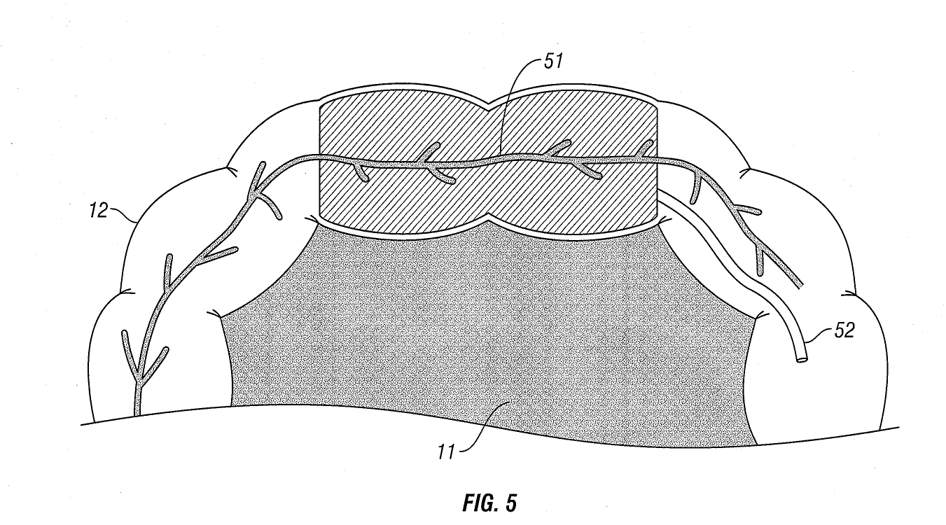

FIG. 5 shows an embodiment that involves the incisionless placement of a tubular or bag-like device 51 via the anal orifice into the rectum. The device may be empty at placement and, following placement, insufflated with a gas, liquid, and/or solid via an insufflation port 52. This insufflation fills the device, expanding it circumferentially as well as proximally into and through the lumen of the rectum and, subsequently, some or all of the colon. When insufflation is terminated, the filled device has formed an intraluminal scaffolding. When a standard laparoscopic instrument is used to grasp the supported segment of bowel wall, the entire supported segment of bowel may be retracted.

In another embodiment, the device may contain a gas, liquid, and/or solid at placement into the rectum and some or all of the colon. Following positioning within the selected segment of bowel, suction may be applied to the device via an aspiration port, creating a rigid intraluminal scaffolding, similar to sand-filled patient positioning blankets, which assume a rigid conformation following suction evacuation. When a standard laparoscopic instrument is used to grasp the supported segment of bowel wall, the entire supported segment of bowel may be retracted.

Other intraluminal scaffolding devices can be provided in accordance with the invention as well. In addition, the use of endoscopy, proctoscopy, sigmoidoscopy, or colonoscopy to position and/or implement function of the intraluminal device may be required. In addition, use of surgical enterotomy with placement of scaffolding devices in the small and/or large bowel may be required.

Common to all intraluminal devices and methods is the creation of a support structure within the bowel lumen which allows for bowel manipulation via laparoscopic instrumentation. The scaffolding, regardless of where the supported segment or segments of bowel is/are grasped by a laparoscopic instrument or instruments, allows for the segment or segments of bowel to be retracted using standard laparoscopic instrumentation. Retraction of a segment or segments of bowel, mimicking retraction used in open surgical procedures, allows for the performance of small and/or large bowel procedures via a laparoscopic approach.

### Laparoscopic Instrumentation Devices and Methods

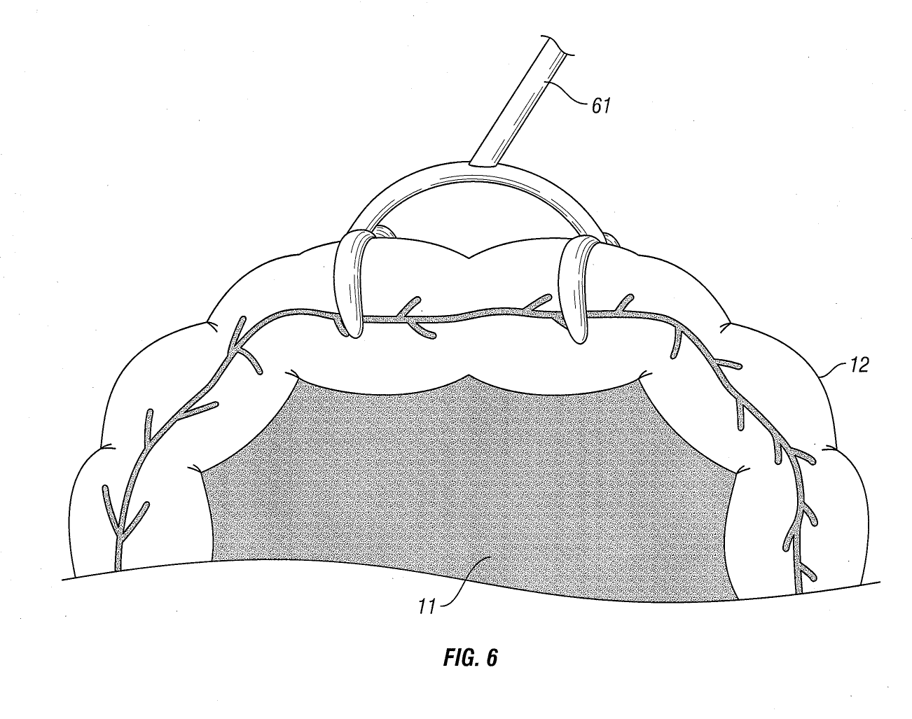

FIG. 6 shows an embodiment of the invention which comprises a laparoscopic instrument 61 which allows for the grasping of bowel wall at two or more locations. The surgeon is thus able to retract a segment of bowel rather than a single point of bowel. Retraction of a segment of bowel, mimicking retraction used in open surgical procedures, allows for the performance of lower small and/or large bowel procedures via a laparoscopic approach. Although the invention is described herein with reference to the preferred embodiment, one skilled in the art will readily appreciate that other applications may be substituted for those set forth herein without departing from the spirit and scope of the present invention. Accordingly, the invention should only be limited by the claims included below.

1. A method for laparoscopic retraction, comprising the steps of:
providing a scaffolding either external to and/or within a selected segment or segments of a patient's gastrointestinal tract for supporting said segment or segments; and
associating with said scaffolding, means for retraction of the entire selected segment or segments via grasping either the scaffolding or the supported segment or segments with a laparoscopic instrument.

2. The method of claim 1, further comprising the step of:
providing a plurality of scaffolding modules for intra-operative assembly.

3. The method of claim 1, further comprising the step of:
passing the components of an extraluminal scaffolding into the patient's abdominal cavity via a laparoscopic or open incisional approach or transluminal or trans-orifice approach.

4. The method of claim 3, further comprising any of the steps of:
providing a ring component for encircling said segment or segments of gastrointestinal tract;
providing a grasping component for grasping said segment or segments of gastrointestinal tract; and
providing an interconnect component for interconnecting and supporting any of said encircling and grasping components.

5. The method of claim 1, further comprising the steps of:
passing a cuff or sleeve into the patient's abdominal cavity via a laparoscopic or open incisional approach or transluminal or trans-orifice approach, wherein said cuff or sleeve is adapted to be placed along a selected segment or segments of said patients' gastrointestinal tract; and
providing a port associated with said cuff or sleeve for receiving infusion of a liquid, gel, or gas, or aspiration of a liquid or gas from said cuff or sleeve, wherein said scaffolding comprises a rigid or semi-rigid structure over a captured segment or segments of said patient's gastrointestinal tract.

6. The method of claim 5, further comprising the step of:
providing flanges for allowing said scaffolding to be grasped by a laparoscopic instrument or instruments.

7. The method of claim 1, comprising the step of:
surrounding the patient's bowel with a support structure comprising means for manipulation of said structure via laparoscopic instrumentation and means for lengthening said structure as required.

8. The method of claim 1, further comprising the step of:
providing a tubular or bag-like device adapted for incisionless insertion into said patient's rectum via the patient's anal orifice or transluminally or via an orifice or via a surgical enterotomy.

9. The method of claim 8, wherein said device is empty at placement and further comprising the step of providing a port for, following placement, insufflation with a gas, liquid, and/or solid, wherein said insufflation fills said device, expanding it circumferentially as well as proximally into and through the lumen of the patient's rectum or transluminally or via an orifice or via a surgical enterotomy and, subsequently, some or all of the patient's colon and/or small intestine, wherein when insufflation is terminated, the filled device comprises an intraluminal scaffolding.

10. The method of claim 9, wherein the device contains a gas, liquid, and/or solid at placement into the patient's rectum or transluminally or via an orifice or via a surgical enterotomy and some or all of the colon and/or small intestine, and further comprising the step of providing an aspiration port wherein, following positioning within the selected segment of organ, suction is applied to the device to create a rigid intraluminal scaffolding.

11. The method of claim 1, further comprising the step of:
a grasping a segment or segments of the patient's gastrointestinal tract with a laparoscopic instrument at two or more locations to retract a segment or segments of said patient's gastrointestinal tract, rather than a single point of said patient's gastrointestinal tract.

12. The method of claim 10, wherein said laparoscopic instrument is adapted for retraction of a segment or segments of bowel, mimicking retraction used in open surgical procedures, to allow for performance of small and/or large bowel procedures via a laparoscopic approach.
